(19) United States
(12) Patent Application Publication
(10) Pub. No.: US 2012/0245465 A1
(43) Pub. Date: Sep. 27, 2012

(54) METHOD AND SYSTEM FOR DISPLAYING INTERSECTION INFORMATION ON A VOLUMETRIC ULTRASOUND IMAGE

(76) Inventors: Joger Hansegard, Oslo (NO); Andreas Michael Ziegler, Oslo (NO); Fredrik Orderud, Oslo (NO)

(21) Appl. No.: 13/072,412

(22) Filed: Mar. 25, 2011

(51) Int. Cl. A61B 8/14; G06T 15/60; G06T 15/08

(52) U.S. Cl. 600/443; 345/419; 345/426

(57) ABSTRACT

A method and system for displaying intersection information on a volumetric ultrasound image are provided. One method includes accessing ultrasound information corresponding to a volume dataset and identifying a location of one or more surfaces intersecting the volume dataset. The method further includes colorizing a rendered image of the volume dataset based on the identified locations of the intersection of the one or more surfaces and displaying a rendered volume dataset with one or more colorized intersections.

### BACKGROUND OF THE INVENTION

The subject matter disclosed herein relates generally to diagnostic ultrasound systems, and more particularly, to a method and system for displaying on a three-dimensional (3D) ultrasound image an intersection with a surface.

When displaying two-dimensional (2D) renderings of 3D volume data, such as in a 3D ultrasound dataset, it may be desirable to visualize one or more surfaces together with the volume data in a manner to allow a visual determination of where the surfaces intersect the volume. For example, it may be desirable to visualize intersections between volume data and planes, intersections between volume data and spheres and other quadric surfaces. In 3D cardiac ultrasound, where it is common to display one or more 2D slice planes reconstructed from a 3D ultrasound data volume, it is important to be able to determine from the displayed information how the 2D slice planes are positioned with respect to the volume rendering to identify the relationship between the two visualization techniques.

Conventional techniques for associating the slice planes with the intersection with the data volume include rendering the plane as a rectangle in space together with the volume. However, with this rectangular plane representation, it can be difficult for the observer to understand precisely where the plane intersects the volume data, which can lead to difficulty in subsequent analysis, such as properly locating smaller anomalies, for example, in the heart valves. Other conventional techniques include displaying an opaque or semi-transparent polygon plane. However, this technique, in addition to the problems described above, also may hide or obscure portions of the volume.

Thus, conventional techniques for identifying the location of a slice plane in an image volume rely on the observer's ability to mentally reconstruct the spatial orientation of the plane based on the shape of the displayed rectangle or plane.

### BRIEF DESCRIPTION OF THE INVENTION

In one embodiment, a method for rendering an ultrasound volume for display is provided. The method includes accessing ultrasound information corresponding to a volume dataset and identifying a location of one or more surfaces intersecting the volume dataset. The method further includes colorizing a rendered image of the volume dataset based on the identified locations of the intersection of the one or more surfaces and displaying a rendered volume dataset with one or more colorized intersections.

In another embodiment, an ultrasound display is provided that includes an image slice display portion displaying one or more two-dimensional (2D) ultrasound image slices. The ultrasound display further includes a volume rendering display portion displaying a rendered three-dimensional (3D) ultrasound image volume having modified visible pixels corresponding to voxels associated with slice planes identified along a surface of the rendered 3D ultrasound image volume. The slice planes correspond to the location of the 2D ultrasound images slices within the 3D ultrasound image volume.

In a further embodiment, an ultrasound system is provided that includes an ultrasound probe configured to acquire a three-dimensional (3D) ultrasound dataset and a signal processor having a surface colorizing module configured to colorize a rendered image of the 3D ultrasound dataset based on identified locations of an intersection of one or more surfaces with the 3D ultrasound dataset. The ultrasound system further includes a display for displaying a rendered volume dataset with one or more colorized intersections.

### BRIEF DESCRIPTION OF THE DRAWINGS

FIG. 1 illustrates a simplified block diagram of an ultrasound system formed in accordance with various embodiments.

FIG. 2 is a flowchart of a method for colorizing intersections between planes and a volume rendering of an ultrasound volume dataset in accordance with various embodiments.

FIG. 3 a block diagram illustrating a rendering process in accordance with one embodiment.

FIG. 4 is a diagram illustrating colorizing of volume samples in accordance with various embodiments.

FIG. 5 is a display of images illustrating colorized intersections displayed in accordance with various embodiments.

FIG. 6 is a block diagram illustrating a rendering process in accordance with another embodiment.

FIG. 7 is a block diagram illustrating a rendering process in accordance with another embodiment.

FIG. 8 are images illustrating colorized intersections displayed in accordance with other various embodiments.

FIG. 9 are curves illustrating transfer functions in accordance with various embodiments.

FIG. 10 is a display of images illustrating colorized intersections displayed in accordance with other various embodiments.

FIG. 11 is a display of images illustrating colorized intersections displayed in accordance with other various embodiments.

FIG. 12 is a block diagram of an ultrasound system formed in accordance with various embodiments.

FIG. 13 is a block diagram of an ultrasound processor module of the ultrasound system of FIG. 12 formed in accordance with various embodiments.

FIG. 14 is a diagram illustrating a three-dimensional (3D) capable miniaturized ultrasound system in which various embodiments may be implemented.

FIG. 15 is a diagram illustrating a 3D capable hand carried or pocket-sized ultrasound imaging system in which various embodiments may be implemented.

FIG. 16 is a diagram illustrating a 3D capable console type ultrasound imaging system in which various embodiments may be implemented.

### DETAILED DESCRIPTION OF THE INVENTION

The foregoing summary, as well as the following detailed description of certain embodiments of the present invention, will be better understood when read in conjunction with the appended drawings. The figures illustrate diagrams of the functional blocks of various embodiments. The functional blocks are not necessarily indicative of the division between hardware circuitry. Thus, for example, one or more of the functional blocks (e.g., processors or memories) may be implemented in a single piece of hardware (e.g., a general purpose signal processor or a block or random access memory, hard disk, or the like) or multiple pieces of hardware. Similarly, the programs may be stand alone programs, may be incorporated as subroutines in an operating system, may be functions in an installed imaging software package, and the like. It should be understood that the various embodiments are not limited to the arrangements and instrumentality shown in the drawings.

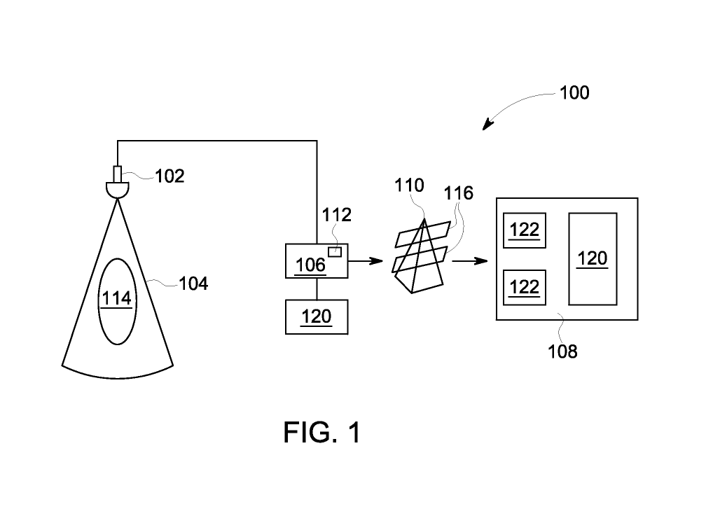

FIG. 1 illustrates a block diagram of an exemplary ultrasound system 100 that is formed in accordance with various embodiments. The ultrasound system 100 includes an ultrasound probe 102 that is used to scan a region of interest (ROI) 104, including one or more objects 114 in the ROI 104. A signal processor 106 processes the acquired ultrasound information received from the ultrasound probe and prepares frames of ultrasound information for display on a display 108. The acquired ultrasound information in one embodiment is a 3D volume dataset 110 that is rendered and displayed on the display 108, for example, in a 3D volume rendering display portion 120. The ultrasound imaging system 100 also includes a surface colorizing module 112 that in some embodiments displays intersection curves on the displayed 3D volume dataset 110 corresponding to the location of one of more surfaces, which are illustrated in this embodiment as slice planes 116. For example, as described in more detail herein, the surface colorizing module 112 uses one or more volume rendering techniques for displaying intersections between the one or more planes 116 (two planes 116 are shown for illustration) and the 3D volume dataset 110. Thus, the volume rendering may be used to visualize where one or more spatial planes intersect the 3D volume dataset 110. In some embodiments, the plane-volume intersection is visualized in the rendering of the 3D volume dataset 110 displayed on the 3D volume rendering display portion 120 by colorizing the image pixels corresponding to the visible voxels that are being intersected, or the voxels that are within a certain distance from the plane(s) 116. It should be noted that the various embodiments are not limited to displaying the intersection between volume data and slice planes. For example, the various embodiments may display the intersections between volume data and spheres and other quadric surfaces. Thus, the various embodiments may be applied to the intersection between the volume data and any geometrical surface.

Accordingly, by colorizing only the visible voxels, the result is a colored intersection curve (e.g., colored line or trace) that appears to be on the surface of the rendered 3D volume dataset 110. Additionally, one or more 2D images 122 corresponding to the one or more slice planes 116 also may be displayed on the display 108. In operation, the colorized intersections may be used, for example, in 3D echocardiography to visualize where a reconstructed 2D ultrasound slice image is positioned in the 3D volume.

At least one technical effect of the various embodiments is providing a visualization of the intersection of a surface with a rendered 3D ultrasound volume. The visualization may be any type of colorizing that is along the surface of the 3D ultrasound volume.

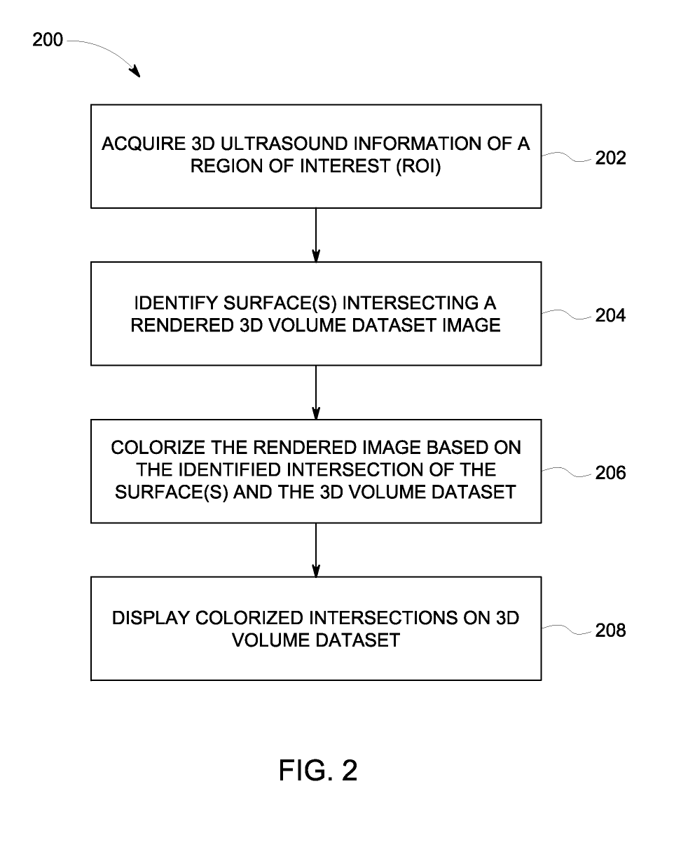

Various embodiments provide a method 200 as shown in the flowchart of FIG. 2 for colorizing one or more intersections between surface(s) and a volume rendering of a 3D ultrasound volume dataset. The method 200 may be embodied as a set of instructions stored on the surface colorizing module 112 shown in FIG. 1. The method 200 may be utilized to visualize, for example, planes or other geometric surfaces on the rendered volume.

The method 200 includes acquiring at 202 ultrasound information of a region of interest (ROI), such as for example the ROI 104 (shown in FIG. 1). In the exemplary embodiment, the ROI 104 is embodied as a structure, such as for example, object 114 shown in FIG. 2, which may be a human heart or region thereof. The ultrasound information may be a volume of data including 3D color Doppler data over time, such as over one or more heart cycles (e.g., ultrasound echocardiography data), and may be stored in a memory device. Optionally, ultrasound information that has been previously acquired and stored in the memory device may be accessed for processing. In one embodiment, the 3D volume dataset 110 is displayed in real-time, for example, on the display 108 at the 3D volume rendering display portion 120 to enable the operator to select one or more surfaces, such as intersecting plane(s), for example, the planes 116 that will be visualized and displayed with corresponding image slices, such as the 2D images 122.

At 204, one or more surfaces are identified that intersect the rendered 3D volume. For example, based on one or more user selected or marked planes, which may be selected image views, a determination is made as to the coordinates of the plane(s) through the 3D volume dataset corresponding to the location in the rendered 3D volume. For example, the operator may manually move or position virtual slices on the screen to selected different views to display. The selection of the one or more slices and the determination of the location of each may be performed using any suitable process or user interface. Thus, in various embodiments, the voxels within the 3D volume dataset corresponding to the user selected plane(s) are determined. The planes may also be located at fixed pre-determined positions relative to the data volume or ultrasound probe. For example, two orthogonal slice planes corresponding to the azimuth and elevation planes of the acquired ultrasound ROI may be positioned such that the planes intersect the center of the data volume. As another example, three slice planes may be rotated about a common axis (such as the probe axis) where the planes are default oriented to provide visualization of a four chamber view, a two chamber view, and a long axis view of the left ventricle of the heart. In these examples, a volume rendering shows the volume data along with the slice intersection curves. The user may or may not modify the position and orientation of these planes.

Thereafter, at 206 the rendered image, for example, the rendered 3D ultrasound volume is colorized based on the identified intersection of the surfaces with the 3D ultrasound volume dataset, which is then displayed with colorized intersection curves at 208. In particular, a parameter of the visible pixels corresponding to the identified voxels is changed such that in various embodiment the selected plane(s) are visible along the surface of the rendered 3D volume, for example, as a curve on the displayed image volume. Any parameter may be changed to identify or highlight the intersection along the surface. For example, the color, transparency, intensity and/or value of the pixels corresponding to the identified intersection voxels may be changed.

In various embodiments, one or more rendering techniques are used for changing a parameter of the pixels in a volume rendering according to where the one or more surfaces intersect the rendered ultrasound data. It should be noted that although the parameter may be described as color, any parameter may be changed or adjusted.

The various embodiments, including the method 200 or the rendering technique 300 described below may be implemented in software, hardware or a combination thereof. For example, the various embodiments for displaying the intersections may be provide on any tangible non-transitory computer readable medium and operate on any suitable computer or processing machine. For example, although the various embodiments may be described in connection with an ultrasound imaging system, the various embodiments may be implemented, on a workstation that does not have ultrasound scanning capabilities. As another example, the various embodiments may be implemented on a system (e.g., an ultrasound system), having a server application that processes data in the background and that can be retrieved or accessed later for display on a client machine. In one embodiment, data is received from an ultrasound scanner and the raw data is converted to rendered Digital Imaging and Communications in Medicine (DICOM) images and stored on a Picture Archiving and Communication System (PACS) device. A user may then retrieve the DICOM images from the device later without use of the various embodiments at that time.

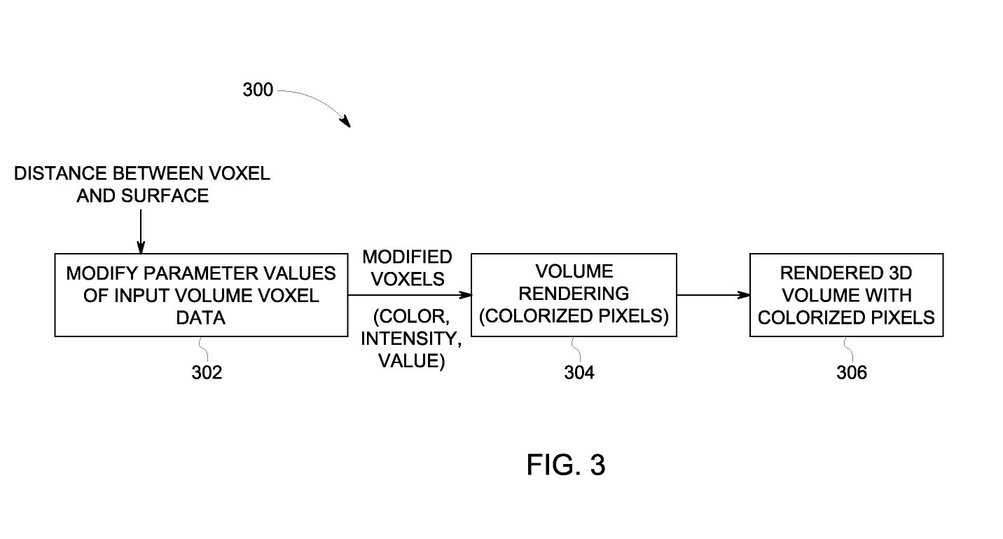

In one embodiment, a rendering technique 300 as shown in FIG. 3 may be used. The rendering technique 300 includes modifying a parameter value of the input volume voxel data prior to rendering. Thus, the input data is changed before volume rendering or updating is performed. In particular, at 302, one or more parameter values, such as the color, intensity and/or value of the input volume samples are changed to reflect the distance between each of the voxel samples and the one or more surfaces (e.g., one or more planes) intersecting the volume. For example, volume elements that are closer to the surface are given a new color, intensity and/or value, whereas volume elements that are at a distance from the surface that is greater than a threshold value (e.g., 3 voxels or a predetermined distance) are unchanged and maintain the current rendered color.

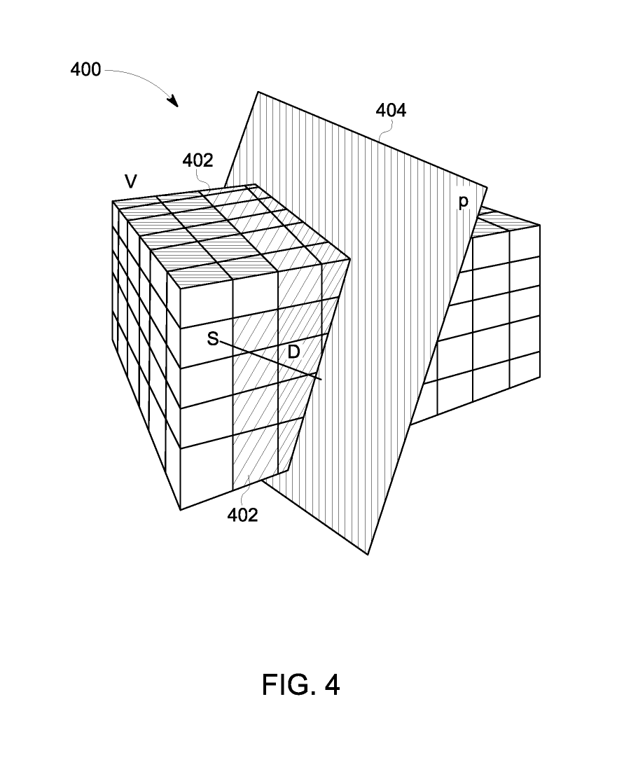

In particular, as shown in FIG. 4, the input volume (V) 400 that is to be rendered includes a small sample of elements sᵢ, where each sample has a coordinate (xᵢ,yᵢ,zᵢ) and a value v(xᵢ,yᵢ,zᵢ). The value may represent a color, an intensity, or any other parameter that is associated with the sample sᵢ. In one embodiment of an ultrasound volume, such as a 3D volume, the samples sᵢ correspond to voxels (volume elements) 402 of the volume 400. In this embodiment, a plane 404, in particular a plane p (a,b,c,d) that intersects the volume 400 is defined by a plane equation as follows:

ax+by+cz+d=0 Equation 1

The signed plane-to-sample distance between the plane 404 and the sample sᵢ can be computed from the coordinate cᵢ, and the plane equation is then defined as follows:

$$D ( {x}_{i} , {y}_{i} , {z}_{i} , p ) = \frac{{ax}_{i} + {by}_{i} + {cy}_{i} + d}{\sqrt{{a}^{2} + {b}^{2} + {c}^{2 }}} \qquad Equation 2$$

Thus, the value V(xᵢ,yᵢ,zᵢ) of each sample (voxel 402) sᵢ is then set in one embodiment based on or according to the distance D between the plane 404 (or other surface) and the sample. For example, every sample having a distance of less than 2 millimeters to the plane 404 can be set to a color, such as red. In some embodiments, the original color of the sample can be modulated using a color transfer function given as M(V(xᵢ,yᵢ,zᵢ), D(xᵢ,yᵢ,zᵢ,p)), which changes the color of the sample depending on the plane-to-sample distance.

Thereafter modified voxels are provided as the input to a volume rendering process 304, which may be any suitable volume rendering process. For example, the input data to the rendering algorithm can be modified by the following (that takes into account multiple planes):

```
for each coordinate (x,y,z) in volume V:
    for each plane p:
        V(x,y,z) = M(V(x,y,z), D(x,y,z,p))
    end for
end for
```

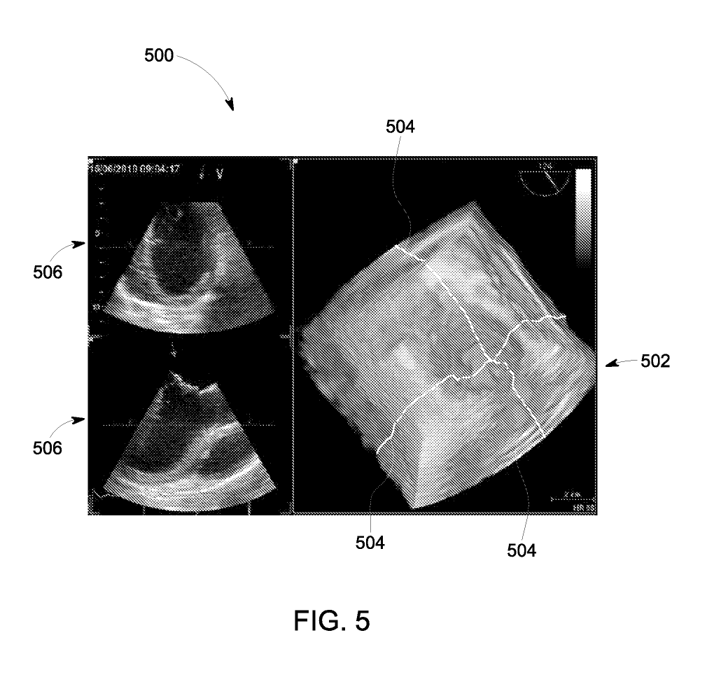

Thus, these modified sample values may be provided to any suitable volume rendering algorithm, with the pixels that represent visible voxels closest to the plane colored accordingly. For example, a rendered 3D volume with colorized pixels may be displayed at 306, such as illustrated in FIG. 5.

In particular, FIG. 5 illustrates an exemplary display 500 having a rendered 3D ultrasound volume 502. As can be seen, two intersection curves 504 (e.g., colored lines) are displayed along the surface of the rendered 3D ultrasound volume 502 corresponding to the planes intersecting the rendered 3D ultrasound volume 502. As can be seen, the curves 504 follow the surface and/or contour of the rendered 3D ultrasound volume 502, and in this embodiment are only displayed along the surface and do not extend above the surface. Additionally, 2D images 506 corresponding to the planes through the rendered 3D ultrasound volume 502 also may be displayed. Thus, the position of one or more 2D slices is displayed as curves 504 in the rendered 3D ultrasound volume 502.

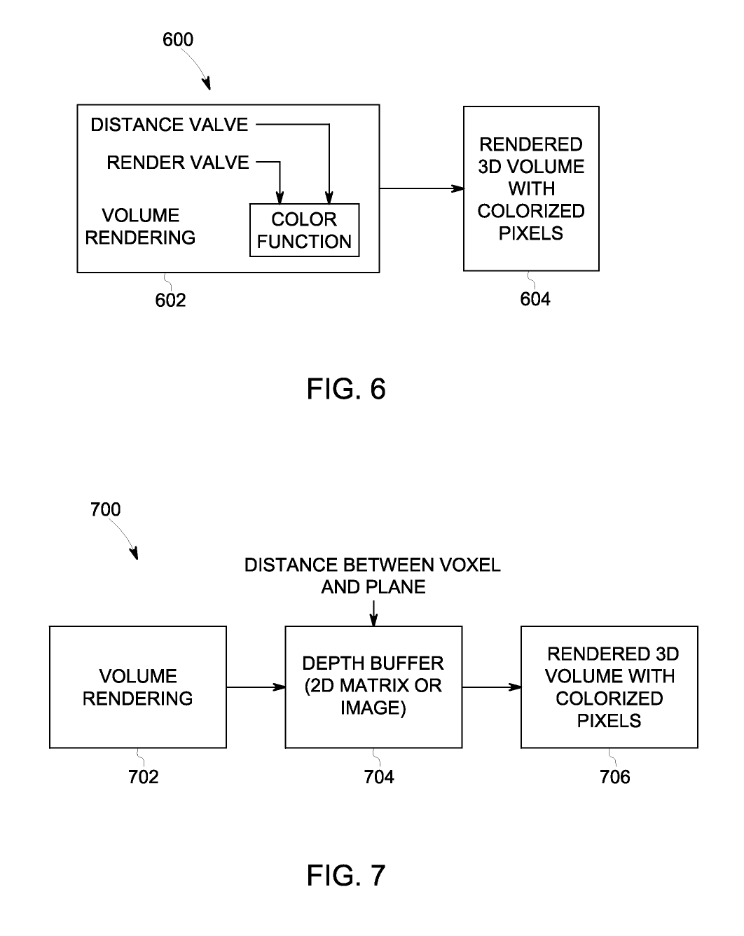

In another embodiment, a rendering technique 600 as shown in FIG. 6 may be used. The rendering technique 600 includes altering a rendering algorithm to modify the color values of the voxels during the rendering. Thus, the color value (or other parameter value) is changed during the rendering process. In particular, at 602 a volume rendering algorithm (e.g., any suitable or conventional rendering process) is modified and used to render a 3D volume with colorized intersections. Specifically, in a volume rendering algorithm, each sample value in the input volume is associated with an opacity value. The opacity value may be computed by applying a transfer function T(V(xᵢ,yᵢ,zᵢ)) to the input sample values.

The rendering algorithm operates by casting rays from a view plane through the data volume, and the volume is sampled at regular intervals along the ray. The rendering is computed as follows:

```
opacity[0] = 1
render_value[0] = 0
for each position (x,y,z) along ray:
    opacity[i] = opacity[i−1] * (1−T(V(x,y,z)))
    render_value[i] = render_value[i−1] + (V(x,y,z) *
    T(V(x,y,z))) * opacity[i−1]
end for
display_value = C(render_value)
```

Thereafter, the output values are mapped to a color using a color transfer function C, and then displayed on a screen at 604 as a rendered 3D volume with colored pixels, such as illustrated in FIG. 5.

In various embodiments, another step is added to the algorithm above where the plane distance is accumulated similarly for the regular sample values as follows:

```
opacity[0] = 1
render_value[0] = 0
dist_value[0] = 0
for each position (x,y,z) along ray:
    opacity[i] = opacity[i−1] * (1−T(V(x,y,z)))
    render_value[i] = render_value[i−1] + V(x,y,z) *
    T(V(x,y,z)) * opacity[i−1]
    for each plane p:
        dist_value[i] = dist_value[i−1] +
        F(D(x,y,z, p))*T(V(x,y,z))*opacity[i−1]
    end for
end for
display_value = C(render_value, dist_value)
```

In this embodiment, F is the transfer function that specifies how fast the color fades away from the plane, such as F(x)=(1−x)³. The color function C has two inputs, namely the render value, and the distance value, and modifies the color depending on the distance value. Thus, in the modified rendering algorithm of this embodiment, the distance values are accumulated the same way as the rendering values, while taking the opacity into account.

In another embodiment, a rendering technique 700 as shown in FIG. 7 may be used. The rendering technique 700 includes modifying the color value (or other parameter value) of pixels in the rendered image based upon a depth buffer after rendering has been performed. In particular, a volume rendering is performed at 702, which may be any suitable volume rendering. One of the outputs of the volume rendering is a depth buffer 704, which is used for colorizing the rendered image, such that a rendered 3D volume with colorized pixels is displayed at 706.

Specifically, in this embodiment, the rendering depth buffer from the rendering algorithm is used for colorization of the rendered image I of volume V after the volume rendering has been performed. The depth buffer (B) 704 is a 2D matrix, or image, wherein the value of each pixel is the depth of each corresponding pixel in the rendered image I. Accordingly, given the coordinates (x,y) of the pixels in I, the depth z of the corresponding pixel is computed from B. The coordinates are then used to calculate the position (x,y,z) of the corresponding sample s in the volume V, such that the sample's distance to the plane is computed to allow for colorization of the rendered image. The depth buffer may be subject to pre-processing steps such as spatial smoothing before computing the sample positions.

In one embodiment, the process or algorithm may be implemented according to the following pseudo-code:

```
tol = tolerance for distance measurement
for each coordinate pair (x,y) in the rendered image I;
    z = B(x,y)
    for each plan p:
        I(x,y) = M(I(x,y), D(x,y,z,p), tol)
    end for
end for
```

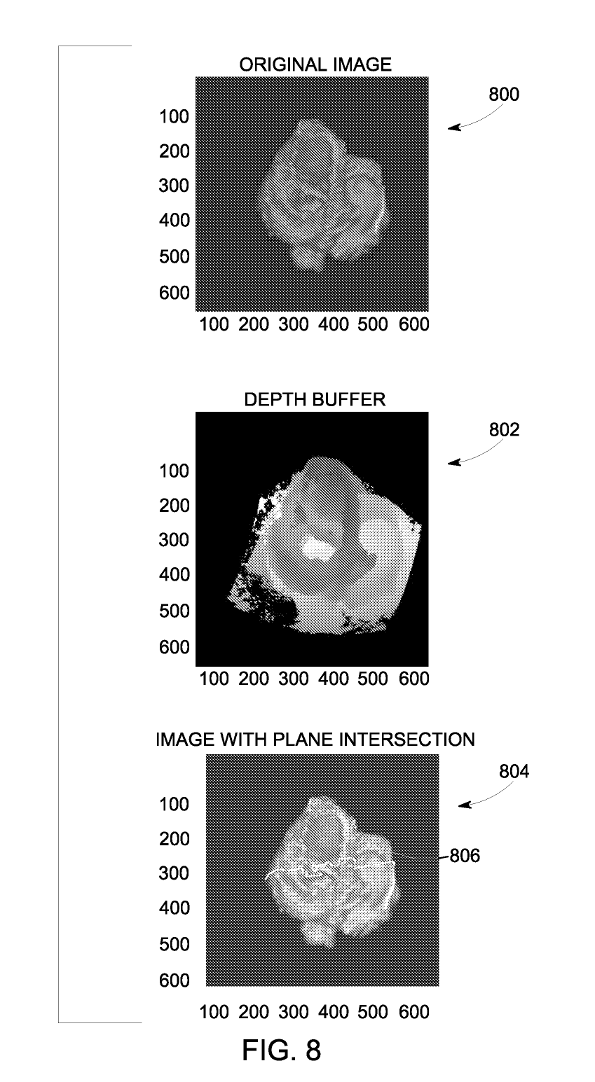

It should be noted that M is a function that modifies the color of the rendered image I based on or according to the distance between the corresponding sample-to-plane distance D and the original value of the rendered image color I(x,y). FIG. 8 illustrates an original image I 800 and the depth buffer B 802. Using these images, a colorized image 804 is generated that includes a line 806 showing the intersection of a plane with the rendered volume.

It also should be noted that in various embodiments the colorization of the volume rendering may be performed in different ways. For example, a single color may be used to colorize the rendered image according to where the plane intersects the volume rendering. As another example, the color may fade away from the line depending on the distance between the corresponding voxel and the plane. Additionally, the color also may be blended with the original color of the volume rendering to provide a semi-transparent appearance for the line.

Thus, in various embodiments, the color transfer function, which may be of the form M(V(xᵢ,yᵢ,zᵢ), d), and which is a function of the value V(xᵢ,yᵢ,zᵢ) of the volume sample s, and the distance d between the plane and the sample, or of the form M(I(x,y), d), which is a function of the value I(x,y) of a rendered image, are used to achieve the colorized rendering that may be modified to provide a desired or required display output.

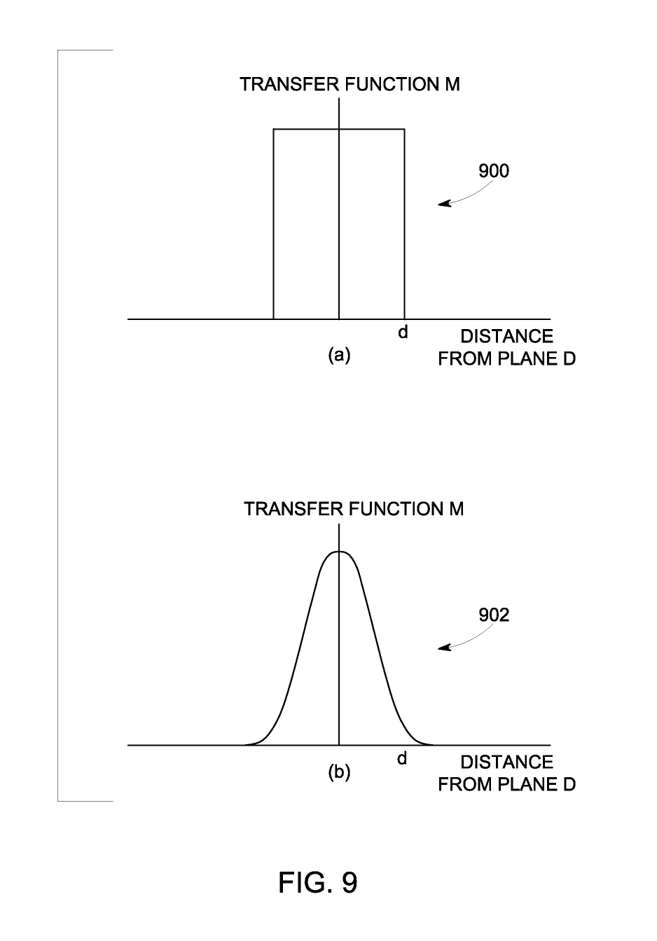

It should be noted that the color transfer function depends on the representation of the sample color, and is application specific in some embodiments. For example, M may just modulate the red channel depending on the plane-to-sample distance, to modify the sample color. In various embodiments, M is a function of D in all color channels, for example, as illustrated in the transfer functions shown in FIG. 9. These transfer functions may be used for colorizing the volume rendering. As illustrated, the transfer function 900 provide a distinct colored line, whereas the transfer function 902 provide a line that gradually fades according to the distance between the plane and the sample.

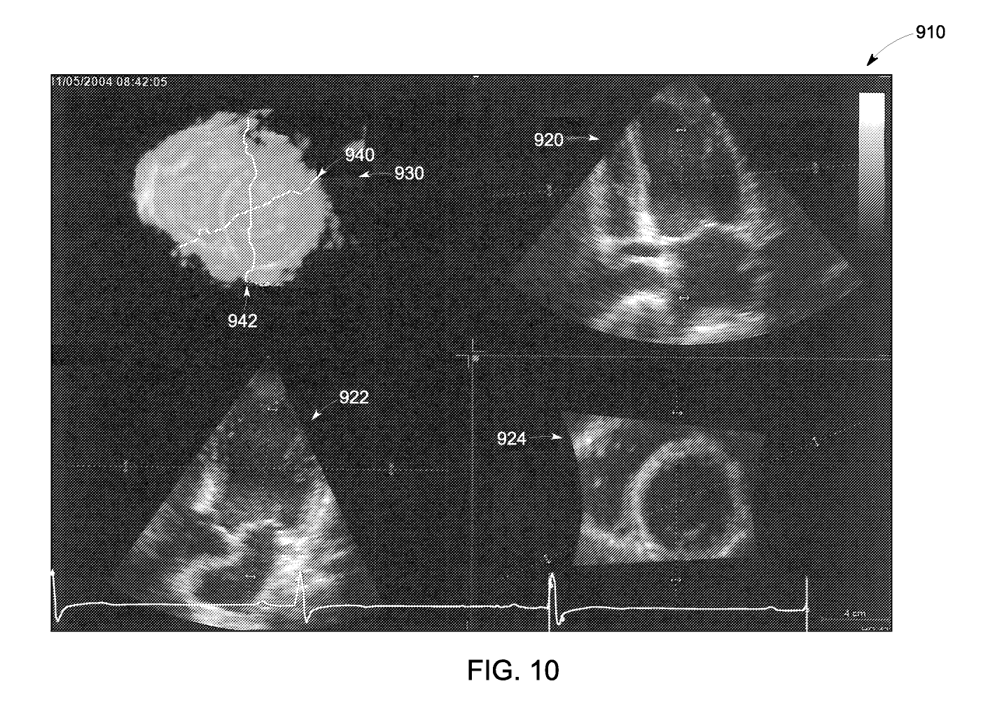

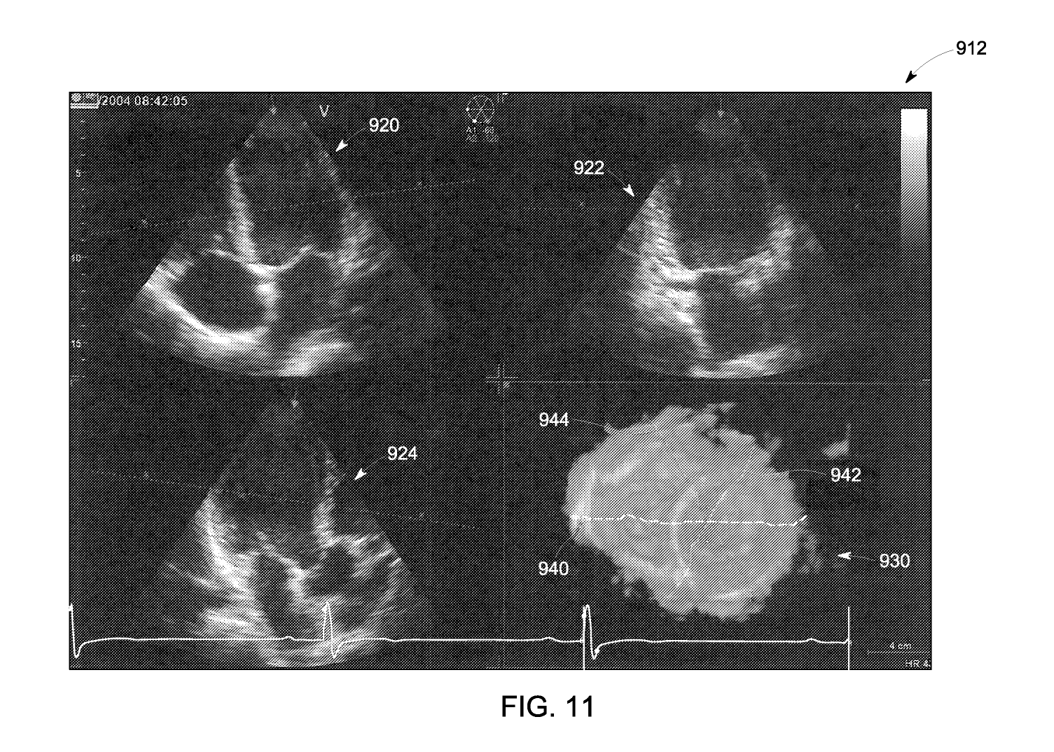

It further should be noted that the color transfer function may also be different for each surface, such as each intersecting plane, such that each plane is colorized in different colors. For example, as illustrated in exemplary display 910 and 912 in FIGS. 10 and 11, respectively, in which three 2D image slices 920, 922 and 924 and one volume rendering 930 (e.g., 3D volume rendering) are reconstructed from the data volume, the different intersection curves (e.g., lines) 940, 942 and 944 (only two are shown in FIG. 10) may be colored differently corresponding to the image slices 920, 922 and 924, respectively. For example, one slice intersection curve may be colored and visualized in white, one in green, and one in yellow. This color coding may be used to provide a visual link between the rendering 930 and the 2D image slices 920, 922 and 924, which may have some associated graphics in the corresponding color, for example, a corresponding colored frame around the slice, color corners, or other visual identifiers.

Thus, various embodiments may provide 3D visualization and navigation having a simplified means to determine the connection or relationship between reconstructed 2D image slices and the corresponding 3D volume rendering.

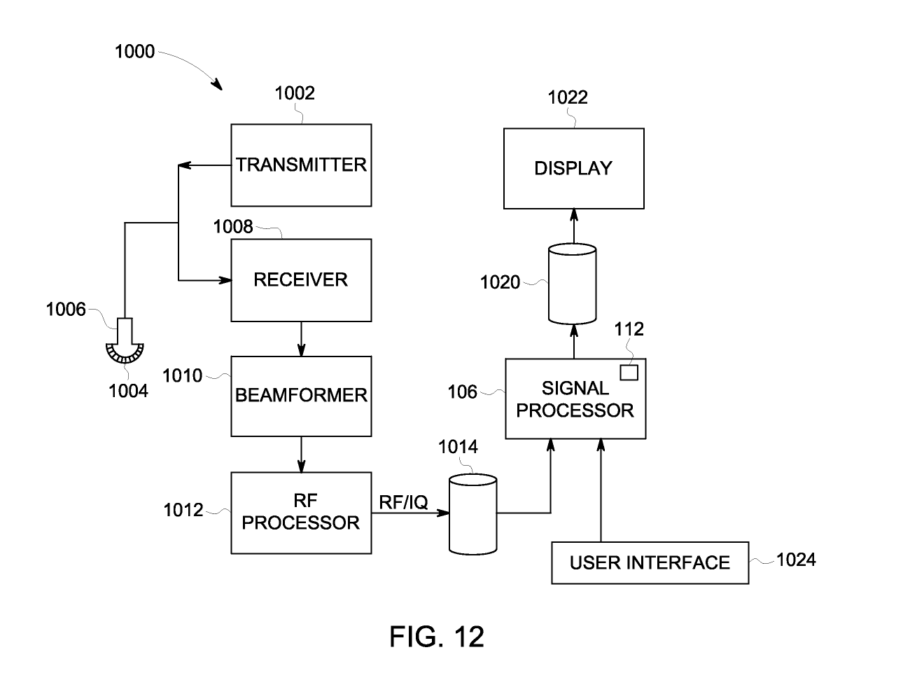

The various embodiments described herein may be implemented in connection with the imaging system shown in FIG. 12. Specifically, FIG. 12 illustrates a block diagram of an exemplary ultrasound system 1000 that is formed in accordance with various embodiments. The ultrasound system 1000 includes a transmitter 1002, which drives a plurality of transducers 1004 within an ultrasound probe 1006 to emit pulsed ultrasonic signals into a body. A variety of geometries may be used. For example, the probe 1006 may be used to acquire 2D, 3D, or 4D ultrasonic data, and may have further capabilities such as 3D beam steering. Other types of probes 1006 may be used. The ultrasonic signals are back-scattered from structures in the body, like blood cells or muscular tissue, to produce echoes which return to the transducers 1004. The echoes are received by a receiver 1008. The received echoes are passed through a beamformer 1010, which performs beamforming and outputs an RF signal. The beamformer may also process 2D, 3D and 4D ultrasonic data. The RF signal then passes through an RF processor 1012. Alternatively, the RF processor 1012 may include a complex demodulator (not shown) that demodulates the RF signal to form IQ data pairs representative of the echo signals. The RF or IQ signal data may then be routed directly to RF/IQ buffer 1014 for temporary storage.

The ultrasound system 1000 also includes a signal processor, such as the signal processor 106 that includes the surface colorizing module 112. The signal processor 106 processes the acquired ultrasound information (i.e., RF signal data or IQ data pairs) and prepares frames of ultrasound information for display on a display 1022. The signal processor 106 is adapted to perform one or more processing operations according to a plurality of selectable ultrasound modalities on the acquired ultrasound information. Moreover, the surface colorizing module 112 is configured to perform the various measurement embodiments described herein. Acquired ultrasound information may be processed in real-time during a scanning session as the echo signals are received. Additionally or alternatively, the ultrasound information may be stored temporarily in the RF/IQ buffer 1014 during a scanning session and processed in less than real-time in a live or off-line operation. A user interface, such as user interface 1024, allows an operator to enter data, enter and change scanning parameters, access protocols, select image slices, and the like. The user interface 1024 may be a rotating knob, switch, keyboard keys, mouse, touch screen, light pen, or any other suitable interface device. The user interface 1024 also enables the operator to reposition or translate the slice planes used to perform measurements as described above.

The ultrasound system 1000 may continuously acquire ultrasound information at a frame rate that exceeds 50 frames per second—the approximate perception rate of the human eye. The acquired ultrasound information, which may be the 3D volume dataset, is displayed on the display 1022. The ultrasound information may be displayed as B-mode images, M-mode, volumes of data (3D), volumes of data over time (4D), or other desired representation. An image buffer (e.g., memory) 1020 is included for storing processed frames of acquired ultrasound information that are not scheduled to be displayed immediately. Preferably, the image buffer 1020 is of sufficient capacity to store at least several seconds worth of frames of ultrasound information. The frames of ultrasound information are stored in a manner to facilitate retrieval thereof according to its order or time of acquisition. The image buffer 1020 may comprise any known data storage medium.

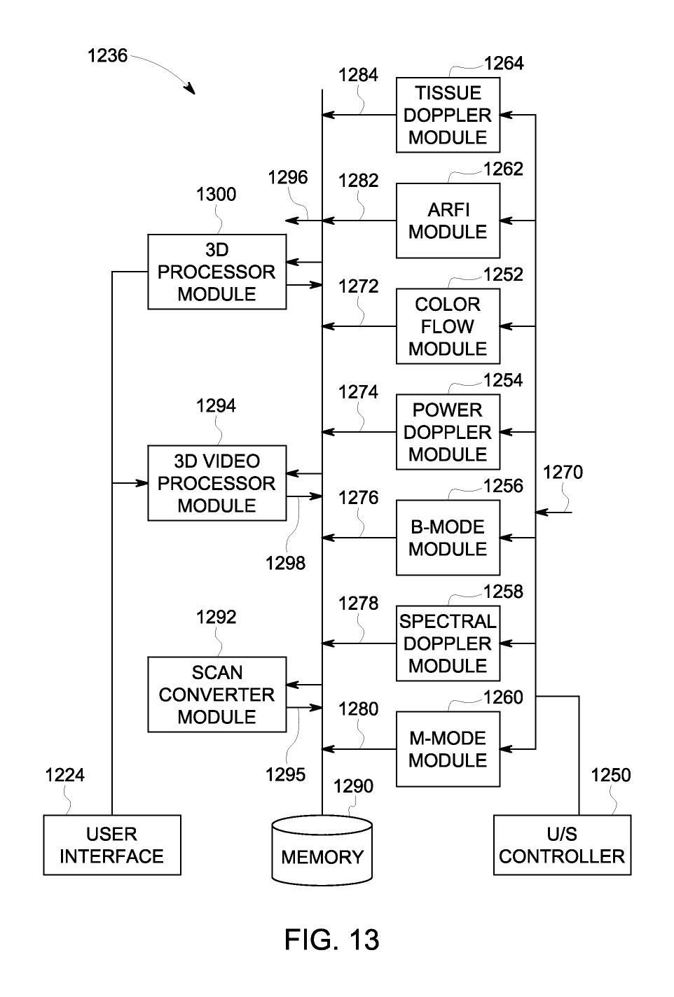

FIG. 13 illustrates an exemplary block diagram of an ultrasound processor module 1236, which may be embodied as the signal processor 106 of FIGS. 1 and 12 or a portion thereof. The ultrasound processor module 1236 is illustrated conceptually as a collection of sub-modules, but may be implemented utilizing any combination of dedicated hardware boards, DSPs, processors, etc. Alternatively, the sub-modules of FIG. 12 may be implemented utilizing an off-the-shelf PC with a single processor or multiple processors, with the functional operations distributed between the processors. As a further option, the sub-modules of FIG. 12 may be implemented utilizing a hybrid configuration in which certain modular functions are performed utilizing dedicated hardware, while the remaining modular functions are performed utilizing an off-the shelf PC and the like. The sub-modules also may be implemented as software modules within a processing unit.

The operations of the sub-modules illustrated in FIG. 13 may be controlled by a local ultrasound controller 1250 or by the processor module 1236. The sub-modules 1252-1264 perform mid-processor operations. The ultrasound processor module 1236 may receive ultrasound data 1270 in one of several forms. In the embodiment of FIG. 11, the received ultrasound data 1270 constitutes I,Q data pairs representing the real and imaginary components associated with each data sample. The I,Q data pairs are provided to one or more of a color-flow sub-module 1252, a power Doppler sub-module 1254, a B-mode sub-module 1256, a spectral Doppler sub-module 1258 and an M-mode sub-module 1260. Optionally, other sub-modules may be included such as an Acoustic Radiation Force Impulse (ARFI) sub-module 1262 and a Tissue Doppler (TDE) sub-module 1264, among others.

Each of sub-modules 1252-1264 are configured to process the I,Q data pairs in a corresponding manner to generate color-flow data 1272, power Doppler data 1274, B-mode data 1276, spectral Doppler data 1278, M-mode data 1280, ARFI data 1282, and tissue Doppler data 1284, all of which may be stored in a memory 1290 (or memory 1014 or memory 1020 shown in FIG. 10) temporarily before subsequent processing. For example, the B-mode sub-module 1256 may generate B-mode data 1276 including a plurality of B-mode image planes, such as in a biplane or triplane image acquisition as described in more detail herein.

The data 1272-1284 may be stored, for example, as sets of vector data values, where each set defines an individual ultrasound image frame. The vector data values are generally organized based on the polar coordinate system.

A scan converter sub-module 1292 accesses and obtains from the memory 1290 the vector data values associated with an image frame and converts the set of vector data values to Cartesian coordinates to generate an ultrasound image frame 1295 formatted for display. The ultrasound image frames 1295 generated by the scan converter module 1292 may be provided back to the memory 190 for subsequent processing or may be provided to the memory 1014 or the memory 1020.

Once the scan converter sub-module 1292 generates the ultrasound image frames 1295 associated with, for example, B-mode image data, and the like, the image frames may be restored in the memory 1290 or communicated over a bus 1296 to a database (not shown), the memory 1014, the memory 1020 and/or to other processors.

The scan converted data may be converted into an X,Y format for video display to produce ultrasound image frames. The scan converted ultrasound image frames are provided to a display controller (not shown) that may include a video processor that maps the video to a grey-scale mapping for video display. The grey-scale map may represent a transfer function of the raw image data to displayed grey levels. Once the video data is mapped to the grey-scale values, the display controller controls the display 1022 (shown in FIG. 12), which may include one or more monitors or windows of the display, to display the image frame. The image displayed in the display 1022 is produced from image frames of data in which each datum indicates the intensity or brightness of a respective pixel in the display.

Referring again to FIG. 13, a 2D video processor sub-module 1294 combines one or more of the frames generated from the different types of ultrasound information. For example, the 2D video processor sub-module 1294 may combine a different image frames by mapping one type of data to a grey map and mapping the other type of data to a color map for video display. In the final displayed image, color pixel data may be superimposed on the grey scale pixel data to form a single multi-mode image frame 1298 (e.g., functional image) that is again re-stored in the memory 1290 or communicated over the bus 1296. Successive frames of images may be stored as a cine loop in the memory 1290 or memory 1020 (shown in FIG. 10). The cine loop represents a first in, first out circular image buffer to capture image data that is displayed to the user. The user may freeze the cine loop by entering a freeze command at the user interface 1224. The user interface 1224 may include, for example, a keyboard and mouse and all other input controls associated with inputting information into the ultrasound system 1000 (shown in FIG. 12).

A 3D processor sub-module 1300 is also controlled by the user interface 1224 and accesses the memory 1290 to obtain 3D ultrasound image data and to generate three dimensional images, such as through volume rendering or surface rendering algorithms as are known. The three dimensional images may be generated utilizing various imaging techniques, such as ray-casting, maximum intensity pixel projection and the like.

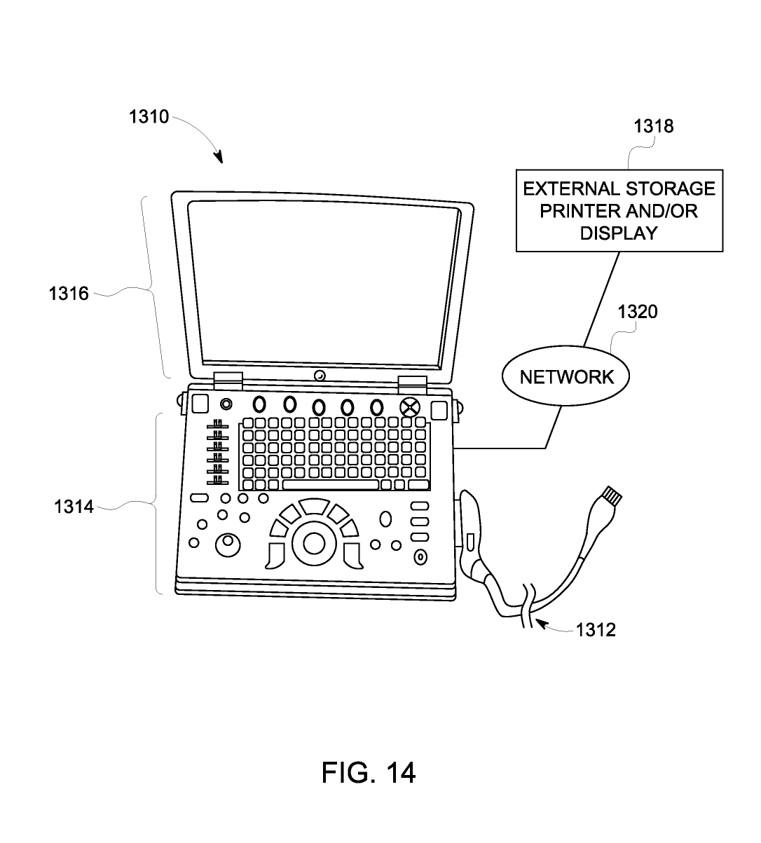

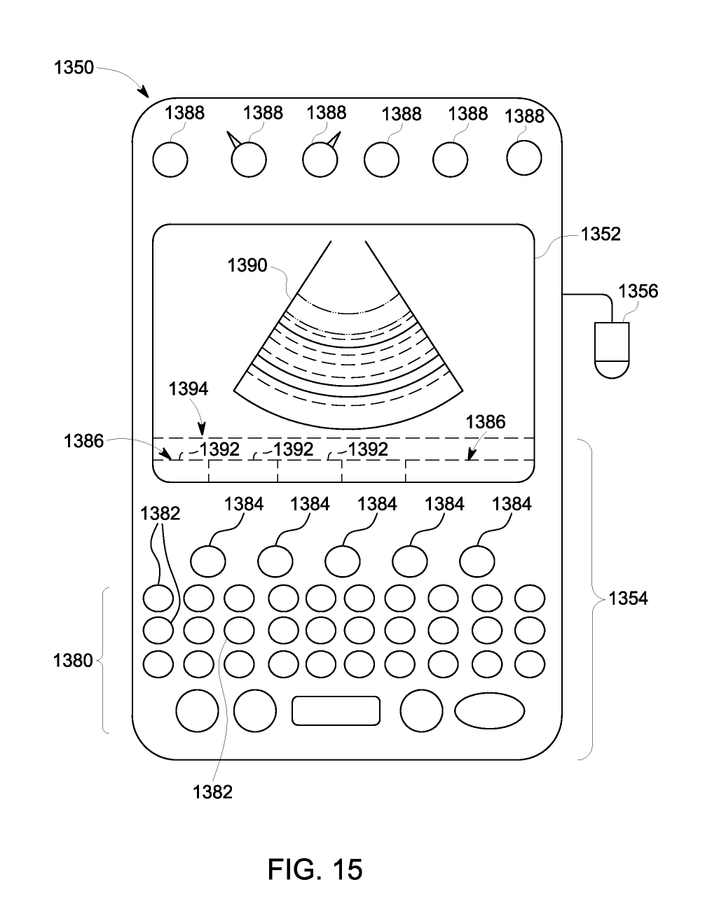

The ultrasound system 1000 of FIG. 12 may be embodied in a small-sized system, such as laptop computer or pocket sized system as well as in a larger console-type system. FIGS. 14 and 15 illustrate small-sized systems, while FIG. 14 illustrates a larger system.

FIG. 14 illustrates a 3D-capable miniaturized ultrasound system 1310 having a probe 1312 that may be configured to acquire 3D ultrasonic data or multi-plane ultrasonic data. For example, the probe 1312 may have a 2D array of transducers 1004 as discussed previously with respect to the probe 1006 of FIG. 12. A user interface 1314 (that may also include an integrated display 316) is provided to receive commands from an operator. As used herein, “miniaturized” means that the ultrasound system 1310 is a handheld or hand-carried device or is configured to be carried in a person's hand, pocket, briefcase-sized case, or backpack. For example, the ultrasound system 1310 may be a hand-carried device having a size of a typical laptop computer. The ultrasound system 1330 is easily portable by the operator. The integrated display 1316 (e.g., an internal display) is configured to display, for example, one or more medical images.

The ultrasonic data may be sent to an external device 1318 via a wired or wireless network 1320 (or direct connection, for example, via a serial or parallel cable or USB port). In some embodiments, the external device 1318 may be a computer or a workstation having a display, or the DVR of the various embodiments. Alternatively, the external device 1318 may be a separate external display or a printer capable of receiving image data from the hand carried ultrasound system 1310 and of displaying or printing images that may have greater resolution than the integrated display 1316.

FIG. 15 illustrates a hand carried or pocket-sized ultrasound imaging system 1350 wherein the display 1352 and user interface 1354 form a single unit. By way of example, the pocket-sized ultrasound imaging system 1350 may be a pocket-sized or hand-sized ultrasound system approximately 2 inches wide, approximately 4 inches in length, and approximately 0.5 inches in depth and weighs less than 3 ounces. The pocket-sized ultrasound imaging system 1350 generally includes the display 1352, user interface 1354, which may or may not include a keyboard-type interface and an input/output (I/O) port for connection to a scanning device, for example, an ultrasound probe 1356. The display 1352 may be, for example, a 320×320 pixel color LCD display (on which a medical image 1390 may be displayed). A typewriter-like keyboard 1380 of buttons 1382 may optionally be included in the user interface 1354.

Multi-function controls 1384 may each be assigned functions in accordance with the mode of system operation (e.g., displaying different views). Therefore, each of the multi-function controls 384 may be configured to provide a plurality of different actions. Label display areas 1386 associated with the multi-function controls 1384 may be included as necessary on the display 1352. The system 1350 may also have additional keys and/or controls 388 for special purpose functions, which may include, but are not limited to “freeze,” “depth control,” “gain control,” “color-mode,” “print,” and “store.”

One or more of the label display areas 1386 may include labels 1392 to indicate the view being displayed or allow a user to select a different view of the imaged object to display. The selection of different views also may be provided through the associated multi-function control 1384. The display 1352 may also have a textual display area 1394 for displaying information relating to the displayed image view (e.g., a label associated with the displayed image).

It should be noted that the various embodiments may be implemented in connection with miniaturized or small-sized ultrasound systems having different dimensions, weights, and power consumption. For example, the pocket-sized ultrasound imaging system 1350 and the miniaturized ultrasound system 1310 may provide the same scanning and processing functionality as the system 1000 (shown in FIG. 12).

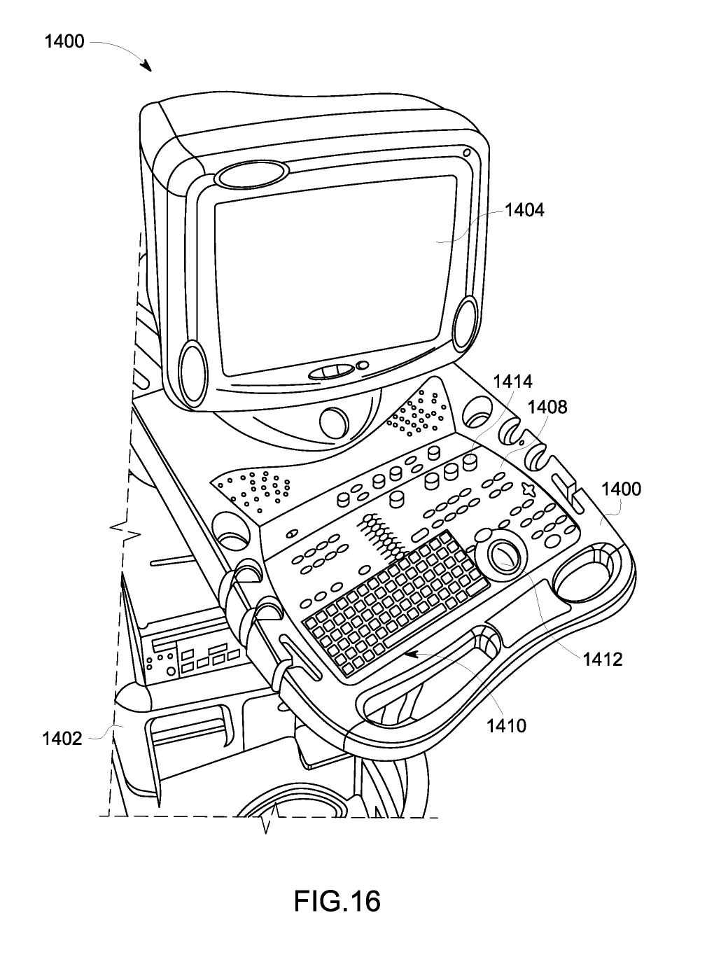

FIG. 16 illustrates an ultrasound imaging system 1400 provided on a movable base 1402. The portable ultrasound imaging system 1400 may also be referred to as a cart-based system. A display 1404 and user interface 406 are provided and it should be understood that the display 1404 may be separate or separable from the user interface 1406. The user interface 1406 may optionally be a touchscreen, allowing the operator to select options by touching displayed graphics, icons, and the like.

The user interface 1406 also includes control buttons 1408 that may be used to control the portable ultrasound imaging system 1400 as desired or needed, and/or as typically provided. The user interface 1406 provides multiple interface options that the user may physically manipulate to interact with ultrasound data and other data that may be displayed, as well as to input information and set and change scanning parameters and viewing angles, etc. For example, a keyboard 1410, trackball 1412 and/or multi-function controls 1414 may be provided.

Exemplary embodiments of an ultrasound system are described above in detail. The ultrasound system components illustrated are not limited to the specific embodiments described herein, but rather, components of each ultrasound system may be utilized independently and separately from other components described herein. For example, the ultrasound system components described above may also be used in combination with other imaging systems.

It should be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, solid state disk drive (e.g., flash drive of flash RAM) and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software. Further, the software may be in the form of a collection of separate programs, a program module within a larger program or a portion of a program module. The software also may include modular programming in the form of object-oriented programming. The processing of input data by the processing machine may be in response to user commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A method for rendering an ultrasound volume for display, the method comprising:
accessing ultrasound information corresponding to a volume dataset;
identifying a location of one or more surfaces intersecting the volume dataset;
colorizing a rendered image of the volume dataset based on the identified locations of the intersection of the one or more surfaces; and
displaying a rendered volume dataset with one or more colorized intersections.

2. The method of claim 1, where the one or more surfaces are a plane.

3. The method of claim 1, where the one or more surfaces are a part of a sphere or other quadric surface.

4. The method of claim 1, wherein the displaying comprises displaying one or more intersection curves along the surface of the rendered volume dataset corresponding to the location of the intersection of the one or more planes with the volume dataset.

5. The method of claim 4, wherein the intersection curve is a colored line and further comprising coloring the line a different color than an original rendered color for the pixels corresponding to the line.

6. The method of claim 5, wherein multiple lines are shown in distinct colors for each of a plurality of surfaces.

7. The method of claim 1, further comprising displaying one or more intersection curves along the surface of the rendered volume dataset corresponding to the location of the intersection of the one or more planes with the volume dataset, and wherein the one or more intersection curves are one of distinct solid colored lines or a colored line fading in color based on a distance from an intersection location of the one or more planes with the volume dataset.

8. The method of claim 1, further comprising modifying a color value of an input volume voxel corresponding to a voxel at one or more of the intersections before rendering the volume dataset.

9. The method of claim 1, further comprising modifying a color value of a voxel at one or more of the intersections during rendering of the volume dataset.

10. The method of claim 1, further comprising estimating distance values within a regular opacity based rendering algorithm, and using a color transfer function for colorizing that accounts for the sample-to-surface distance.

11. The method of claim 1, further comprising modifying a color of pixels in the rendered volume dataset corresponding to the one or more intersections after image rendering and based on a depth buffer determined during the image rendering.

12. The method of claim 11, where the depth buffer is spatially filtered.

13. The method of claim 11, wherein the depth buffer comprises a two-dimensional matrix of depth values.

14. The method of claim 1, further comprising displaying image slices corresponding to the one or more intersections with the rendered volume dataset.

15. The method of claim 1, further comprising changing one of an intensity or a value of colorized pixels corresponding to voxels intersected by the one or more planes.

16. An ultrasound display comprising:
an image slice display portion displaying one or more two-dimensional (2D) ultrasound image slices; and
a volume rendering display portion displaying a rendered three-dimensional (3D) ultrasound image volume having modified visible pixels corresponding to voxels associated with slice planes identified along a surface of the rendered 3D ultrasound image volume, wherein the slice planes correspond to the location of the 2D ultrasound images slices within the 3D ultrasound image volume.

17. The ultrasound display of claim 16, wherein the modified visible pixels form a visible curve along the surface of the rendered 3D ultrasound image volume corresponding to an intersection of the slice planes with the surface.

18. The ultrasound display of claim 17, wherein the curve follows the contour of the rendered 3D ultrasound image volume.

19. The ultrasound display of claim 17, wherein the curve is one of a distinct solid colored line having a changed color with respect to a rendered color or a colored line having fading color based on a distance from an intersection location of the one or more slice planes with the surface of the rendered 3D ultrasound image volume.

20. The ultrasound display of claim 16, wherein the image slices correspond to image planes.

21. The ultrasound display of claim 16, wherein the image slices correspond to a geometric surface that is part of a sphere of other quadric surface.

22. The ultrasound display of claim 16, wherein the modified visible pixels corresponding to voxels associated with slice planes identified along a surface of the rendered 3D ultrasound image volume are a different color for each of the 2D ultrasound images slices.

23. An ultrasound system comprising:
an ultrasound probe configured to acquire a three-dimensional (3D) ultrasound dataset;
a signal processor having a surface colorizing module configured to colorize a rendered image of the 3D ultrasound dataset based on identified locations of an intersection of one or more surfaces with the 3D ultrasound dataset; and
a display for displaying a rendered volume dataset with one or more colorized intersections.

24. The ultrasound system of claim 23, wherein the surface colorizing module is configured to generate an intersection curve for display along a surface of the rendered volume dataset.

25. The ultrasound system of claim 23, wherein the surface comprises a geometric shape being one or more of a plane, a part of a sphere or a quadric surface.

26. The ultrasound system of claim 23, wherein each surface corresponding to the identified locations is a colorized intersection having a different color than the other colorized intersections.
